(19) United States
(12) Patent Application Publication
(10) Pub. No.: US 2026/0155210 A1
(43) Pub. Date: Jun. 4, 2026

(54) METHODS, SYSTEMS, DEVICES, AND MEDIA FOR SCREENING MARKERS FOR DIAGNOSING CANCER BASED ON METHYLATED CFDNA FRAGMENTS

(71) Applicant: ONKOCARE LIFE TECH. (SUZHOU) CO., LTD, Suzhou (CN)

(72) Inventors: Qianghu WANG, Suzhou (CN); Lingxiang WU, Suzhou (CN); Ruohan ZHANG, Suzhou (CN)

(73) Assignee: ONKOCARE LIFE TECH. (SUZHOU) CO., LTD, Suzhou (CN)

(21) Appl. No.: 19/383,827

(22) Filed: Nov. 10, 2025

Related U.S. Application Data
(63) Continuation of application No. PCT/CN2024/144372, filed on Dec. 31, 2024.

(30) Foreign Application Priority Data
Jun. 3, 2024 (CN) 202410704131.1

(51) Int. Cl. G16B 30/10; G16B 40/00

(52) U.S. Cl. CPC G16B 30/10; G16B 40/00

(57) ABSTRACT

The present disclosure relates to a method, a system, a device, and a media for markers for diagnosing cancer based on methylated cfDNA fragments. The method and system of the present disclosure are capable of simultaneously identifying genomic window features, fragment features, and terminal sequences features to provide new biomarkers and targets for disease diagnosis and treatment.

### CROSS-REFERENCE TO RELATED APPLICATIONS

This application is a Continuation of International Application No. PCT/CN2024/144372, filed on Dec. 31, 2024, which claims priority to the Chinese Patent Application No. 202410704131.1, filed on Jun. 3, 2024, the contents of which are hereby incorporated by reference.

### TECHNICAL FIELD

The present disclosure relates to the technical field of screening and application of cancer markers, and specifically, to a method, a system, a device, and a medium for markers for diagnosing cancer based on methylated cfDNA fragments.

### BACKGROUND

With the advancement of biomedical research, the demand in the field of tumor liquid biopsy for detecting DNA methylation level, insert fragment size, and terminal sequence is becoming increasingly urgent. DNA methylation level refers to the addition and removal of a methyl group on a DNA molecule, which plays an important role in gene expression regulation, cellular differentiation, and disease development. Insert fragment size usually refers to the length of a DNA fragment obtained by shearing DNA molecules in a sample using sonication or enzymatic digestion during library construction for next-generation sequencing. The terminal sequence refers to a sequence of a certain length at both ends of a DNA molecule.

Currently, existing manners for DNA methylation detection include bisulfite sequencing manners such as WGBS and RRBS. However, bisulfite conversion of DNA has two serious drawbacks: great DNA loss and destruction of the original fragment structure of DNA. This prevents bisulfite sequencing data of cfDNA from capturing insert fragment size and terminal sequence information. Therefore, a distribution of the insert fragment size and the terminal sequences is generally assessed using WGS data, making the detection process cumbersome.

### SUMMARY

A first aspect of the present disclosure provides a method for screening markers for diagnosing cancer based on methylated cfDNA fragments. The method includes the following steps.
S1, obtaining fragment sequence information and methylation information of cfDNA samples from cancer patients and cfDNA samples from normal subjects; the methylation information includes methylation positions and methylation levels;
S2, performing the following analyses on the fragment sequence information and the methylation information obtained in the step S1 to obtain a candidate marker level profile: (a) dividing a whole human genome by length to obtain a plurality of different genomic windows, determining a count of methylated cfDNA fragments aligned to each of the plurality of different genomic windows, and obtaining a genomic window methylation level profile based on the methylation information and the count of methylated cfDNA fragments; (b) determining frequencies of fragment features with different step sizes based on counts of methylated cfDNA fragments of different lengths to obtain a fragment feature frequency profile; and (c) determining distribution frequencies of 4mer to 6mer terminal sequences at 5′ end of the methylated cfDNA fragments to obtain a terminal sequence frequency profile;

The candidate marker level profile includes the genomic window methylation level profile, the fragment feature frequency profile, and the terminal sequence frequency profile; and
S3, screening candidate markers that exhibit significant differences between the cancer patients and the normal subjects based on the candidate marker level profile obtained in the step S2, to determine markers for diagnosing cancer, including genomic windows, fragment features, and terminal sequences.

A second aspect of the present disclosure provides a system for screening markers for diagnosing cancer based on methylated cfDNA fragments, including:
A methylation data input module, configured to receive the fragment sequence information and the methylation information of the cfDNA samples from the cancer patients and the cfDNA samples from the normal subjects;
A genomic data storage module, configured to store whole human genome data;
A data alignment module, connected to the methylation data input module and the genomic data storage module, respectively, and configured to align the fragment sequence information and the methylation information with the whole human genome data;
An analysis module, connected to the data alignment module, and configured to perform the following analyses to obtain a candidate marker level profile:
(a) dividing the whole human genome by length to obtain a plurality of different genomic windows, determining a count of methylated cfDNA fragments aligned to each of the plurality of different genomic windows, and obtaining a genomic window methylation level profile based on the methylation information and the count of methylated cfDNA fragments;
(b) determining frequencies of fragment features with different step sizes based on counts of methylated cfDNA fragments of different lengths to obtain a fragment feature frequency profile; and
(c) determining distribution frequencies of 4mer to 6mer terminal sequences at 5′ end of the methylated cfDNA fragments to obtain a terminal sequence frequency profile;

The candidate marker level profile includes the genomic window methylation level profile, the fragment feature frequency profile, and the terminal sequence frequency profile; and

A screening module, connected to the analysis module, and configured to screen candidate markers that exhibit significant differences between the cancer patients and the normal subjects based on the candidate marker level profile.

A third aspect of the present disclosure provides a marker combination for diagnosing cancer, including markers screened by the method according to the first aspect of the present disclosure or the system according to the second aspect of the present disclosure.

A fourth aspect of the present disclosure provides another marker combination for diagnosing cancer, including:
(a) at least one from a genomic window combination consisting of chr15: 31775701-31776000, chr7: 32467501-32467800, and chr8: 24771301-24771600;
(b) at least one from a fragment feature combination consisting of 165-166 bp, 163-165 bp, and 167-168 bp; and
(c) at least one from a terminal sequence combination consisting of ATGGGG, ATAGGC, and ATGAGG;
   the cancer is colorectal cancer;
   including:
(a) at least one from a genomic window combination consisting of chr7: 32467501-32467800, chr7: 32467801-32468100, and chr20: 21376801-21377100;
(b) at least one from a fragment feature combination consisting of 149-150 bp, 151-152 bp, and 153-154 bp; and
(c) at least one from a terminal sequence combination consisting of ATGAGC, ATGG, and ATAGCG;
   the cancer is liver cancer;
   including:
(a) at least one from a genomic window combination consisting of chr20: 43726801-43727100, chr6: 107955901-107956200, and chr19: 19650901-19651200;
(b) at least one from a fragment feature combination consisting of 279-280 bp, 277-279 bp, and 280-282 bp; and
(c) at least one from a terminal sequence combination consisting of CTAGGG, TTCAGC, and ATAGGC;
   the cancer is pancreatic cancer;
   including:
(a) at least one from a genomic window combination consisting of chr7: 32467501-32467800, chr6: 107955901-107956200, and chr2: 39187201-39187500;
(b) at least one from a fragment feature combination consisting of 163-164 bp, 165-166 bp, and 156-160 bp; and
(c) at least one from a terminal sequence combination consisting of AGGGAG, AGGGGA, and TGAAAC;
   the cancer is gastric cancer; or
   including:
(a) at least one from a genomic window combination consisting of chr3: 51740701-51741000, chr9: 140128201-140128500, and chr17: 7670401-7670700;
(b) at least one from a fragment feature combination consisting of 267-268 bp, 259-260 bp, and 268-270 bp; and
(c) at least one from a terminal sequence combination consisting of GGGAAC, GGGAGT, and GGGAAG;
   the cancer is lung cancer.

A fifth aspect of the present disclosure provides for an application of a detection reagent and/or a device for the marker combination according to the third or fourth aspect of the present disclosure in preparing a kit for diagnosing cancer.

A sixth aspect of the present disclosure provides a computer device including: a memory, configured to store a computer program; and a processor, configured to execute the computer program to implement the method for screening markers for diagnosing cancer based on methylated cfDNA fragments according to the first aspect of the present disclosure.

A seventh aspect of the present disclosure provides a non-transitory computer-readable storage medium. The computer-readable storage medium stores a computer program, and the computer program is executed by a processor to implement the method for screening markers for diagnosing cancer based on methylated cfDNA fragments according to the first aspect of the present disclosure.

An eighth aspect of the present disclosure provides a system for diagnosing cancer including: a marker data input module, configured to input levels of a plurality of markers in the marker combination according to the third aspect of the present disclosure or the fourth aspect of the present disclosure; and a cancer judgment module, connected to the marker data input module, and configured to judge whether a subject has cancer or is at risk of having cancer based on the levels of the plurality of markers in the marker combination.

### BRIEF DESCRIPTION OF THE DRAWINGS

The disclosure will be further illustrated by way of exemplary embodiments, which will be described in detail by means of the accompanying drawings.

FIG. 1 is a schematic flow diagram illustrating a method for screening markers for diagnosing cancer based on sequencing of methylated cfDNA fragments according to Embodiment 1 of the present disclosure.

FIG. 2 is a schematic diagram illustrating distribution frequencies of insert fragment size obtained through fragment size analysis according to Embodiment 1 of the present disclosure.

FIG. 3 is a schematic diagram illustrating a system for screening markers for diagnosing cancer based on sequencing of methylated cfDNA fragments according to Embodiment 1 of the present disclosure.

FIG. 4 is a schematic diagram illustrating box plots showing distributions of a methylation level feature chr15: 31775701-31776000, a fragment feature 165-166 bp, and a terminal sequence feature ATGGGG highly enriched in the colorectum, in colorectal cancer samples and normal subject samples, and ROC curves illustrating an ability of the three features to distinguish the colorectal cancer samples from the normal subject samples.

FIG. 5 is a schematic diagram illustrating box plots showing distributions of a methylation level feature chr7: 32467501-32467800, a fragment feature 163-165 bp, and a terminal sequence feature ATAGGC highly enriched in the colorectum, in colorectal cancer samples and normal subject samples, and ROC curves illustrating an ability of the three features to distinguish the colorectal cancer samples from the normal subject samples.

FIG. 6 is a schematic diagram illustrating box plots showing distributions of a methylation level feature chr8: 24771301-24771600, a fragment feature 167-168 bp, and a terminal sequence feature ATGAGG highly enriched in the colorectum, in colorectal cancer samples and normal subject samples, and ROC curves illustrating an ability of the three features to distinguish the colorectal cancer samples from the normal subject samples.

### DETAILED DESCRIPTION

Unless otherwise indicated, implied by the context, or consistent with conventional practice in the prior art, all counts and percentages in the present disclosure are by weight, and all testing and characterization manners are those in effect as of the filing date of the present disclosure. Where applicable, any patents, patent applications, or publications referred to in the present disclosure are incorporated herein by reference in their entirety, and equivalent family patents of the referenced patents, patent applications, or publications are also incorporated by reference, particularly with respect to the definitions of relevant terms in the field disclosed in the referenced documents. In the event that the definitions of specific terms disclosed in the prior art are inconsistent with any definitions provided in the present disclosure, the definitions provided in the present disclosure shall prevail.

In order to make the technical problems, technical solutions, and beneficial effects addressed by the present disclosure clearer, the present disclosure is further described in detail below in conjunction with the embodiments.

The following embodiments are provided herein to illustrate preferred embodiments of the present disclosure. It should be understood by those skilled in the art that the technologies disclosed in the following embodiments represent technologies discovered by the inventors that may be used to implement the present disclosure, and therefore may be regarded as preferred embodiments of the present disclosure. It should be understood by those skilled in the art that, based on the present disclosure, specific embodiments disclosed herein may be modified in many ways while still achieving the same or similar results, without departing from the spirit or scope of the present disclosure.

Unless otherwise defined, all technical and scientific terms used herein have the same meanings as commonly understood by those skilled in the art to which the present disclosure belongs, and all references disclosed herein, together with materials cited therein, are incorporated herein by reference.

Those skilled in the art will recognize, or be able to ascertain through routine experimentation, many equivalent technologies of the specific embodiments of the present disclosure described herein. The equivalent technologies will be encompassed in claims.

In some embodiments, the present disclosure provides a method for screening markers for diagnosing cancer based on methylated cfDNA fragments, including steps as follows:
S1, obtaining fragment sequence information and methylation information of cfDNA samples from cancer patients and cfDNA samples from normal subjects; the methylation information includes methylation positions and methylation levels;
S2, performing the following analyses on the fragment sequence information and the methylation information obtained in the step S1 to obtain a candidate marker level profile: (a) dividing a whole human genome by length to obtain a plurality of different genomic windows, determining a count of methylated cfDNA fragments aligned to each of the plurality of different genomic windows, and obtaining a genomic window methylation level profile based on the methylation information and the count of methylated cfDNA fragments; (b) determining frequencies of fragment features with different step sizes based on counts of methylated cfDNA fragments of different lengths to obtain a fragment feature frequency profile; and (c) determining distribution frequencies of 4mer to 6mer terminal sequences at 5′ end of the methylated cfDNA fragments to obtain a terminal sequence frequency profile; the candidate marker level profile includes the genomic window methylation level profile, the fragment feature frequency profile, and the terminal sequence frequency profile; and
S3, screening candidate markers that exhibit significant differences between the cancer patients and the normal subjects based on the candidate marker level profile obtained in the step S2, to determine markers for diagnosing cancer, including genomic windows, fragment features, and terminal sequences.

In the present disclosure, the cfDNA is referred to as “cell-free DNA”, i.e., cell free DNA, and is derived from sources including, but not limited to, peripheral blood, cerebrospinal fluid, saliva, pleural fluid, ascites, urine, and feces.

The fragment sequence information refers to raw sequence data of a DNA fragment directly obtained by high-throughput sequencing technology. The fragment sequence information includes a base sequence, a sequencing quality value, and a fragment length (in base pairs, bp).

The methylation information refers to information about a cfDNA fragment that has undergone methylation. The methylation information includes the methylation positions and the methylation levels. The methylation position refers to a coordinate of a specific cytosine (C) base in the reference genome where DNA methylation modification occurs. The methylation level, also referred to as a degree of methylation, is a quantitative measure that describes a proportion of DNA molecules methylated at a specific methylation site (CpG site) relative to a total count of DNA molecules. The methylation level (at a given site)=(count of methylated reads)/(count of methylated reads+count of unmethylated reads)×100%.

In some embodiments, the fragment sequence information and the methylation information are obtained by (a) obtaining the methylated cfDNA fragments of a sample to be tested by means of methylation-enriched protein enrichment, immunoprecipitation, or enzymatic conversion, and (b) sequencing the methylated cfDNA fragments to obtain methylation sequencing data. This manner captures the cfDNA that undergoes methylation while retaining fragment features of the cfDNA well enough for subsequent simultaneous detection of the cfDNA methylation level, the insert fragment size, and the terminal sequence.

In some embodiments, between the step S1 and the step S2, a step of preprocessing the fragment sequence information is included. The preprocessing includes:
(a) removing adapter sequences;
(b) filtering out low-quality sequences in which more than 40% of bases have a quality score lower than Q15;
(c) filtering out sequences containing more than 5 bases of N;
(d) filtering out sequences with a length shorter than 30 (overly short sequences); and
(e) trimming four bases at the fragment ends with an average quality score lower than Q20.

In some embodiments, a methylated standard and an unmethylated standard are added to both the cfDNA samples from the cancer patients and the cfDNA samples from the normal subjects. In some embodiments, the methylated standard is a fully methylated positive control ADNA, and the unmethylated standard refers to a fully unmethylated negative control ADNA.

Furthermore, preprocessed sequencing data (i.e., the fragment sequence information) is aligned using Bowtie2-2.3.4.2 to a human reference genome hg19 (GRCh37) and to a ADNA reference genome to generate BAM files. The BAM files are then sorted according to genomic coordinates, deduplicated using Picard MarkDuplicates-2.18.25-SNAPSHOT, and finally screened to retain paired reads that are both aligned to the reference genomes with MAPQ>20.

According to the alignment results of the fully methylated positive control λDNA and the fully unmethylated negative control λDNA, a reaction specificity rate is calculated. The reaction specificity rate refers to a ratio of a count of reads aligned to the fully methylated positive control λDNA to a sum of the reads aligned to the fully methylated positive control λDNA and the fully unmethylated negative control λDNA, as described in Section 4 of Embodiment 1. In some embodiments, in the step S1, methylation sequencing data with an effective sequencing data yield greater than a first preset threshold and the reaction specificity rate greater than a second preset threshold are selected, and the fragment sequence information and the methylation information are determined based on the selected methylation sequencing data. The effective sequencing data yield refers to a total count of reads aligned to the human reference genome after deduplication and screening. The first preset threshold may range from 4 G to 9 G, and the second preset threshold may range from 0.7 to 0.9. If no methylation sequencing data from the cfDNA samples from the cancer patients or from the cfDNA samples from the normal subjects meet the requirements, library preparation and sequencing are repeated, or new samples are obtained for library preparation and sequencing.

In some embodiments, in the step S2, the genomic window methylation level profile is obtained based on steps as follows: dividing the whole human genome by length to obtain the plurality of different genomic windows; for each genomic window among the plurality of different genomic windows, determining a count of methylated cfDNA fragments within the genomic window; and normalizing the count of methylated cfDNA fragments to obtain a methylation level of the genomic window. A plurality of methylation levels corresponding to the plurality of different genomic windows constitute the genomic window methylation level profile.

In some embodiments, normalized methylation levels are obtained using the following formula:

$$FPKM = \frac{count of methylated cfDNA fragments within the genomic window}{total count of methylated cfDNA fragments \times the genomic window length}$$

A total count of methylated cfDNA fragments is expressed in millions, and the genomic window length is expressed in kilobases (kb). The total count of methylated cfDNA fragments refers to a total count of all methylated cfDNA fragments in the plurality of genomic windows.

In some embodiments, when the windows are defined with a length of 300 bp, the whole human genome may be divided into 10,318,991 genomic windows. Those skilled in the art may select different lengths for window definition, and may also perform exhaustive division within a range from 1 to a size of the genome.

In the present disclosure, the “fragment feature”, also referred to as “insert fragment size feature”, refers to all methylated cfDNA fragments within each fragment interval after the methylated cfDNA fragments are divided into different fragment intervals according to different base pair lengths (i.e., step sizes). Each fragment interval includes all methylated cfDNA fragments of the corresponding fragment lengths. The fragment feature is represented as intervals of fragment lengths, for example, 61-70 bp, 74-75 bp, etc. In some embodiments, the step size is 2 bp to 10 bp. For example, when the step size is 2 bp, the divided fragment intervals are 61-62 bp, 63-64 bp, . . . , 399-400 bp. When the step size is 3 bp, the divided fragment intervals are 61-63 bp, 64-66 bp, . . . , and 397-399 bp. When the step size is 10 bp, the divided fragment intervals are 61-70 bp, 71-80 bp, . . . , 391-400 bp. For example, if the fragment feature is 61-65 bp, the fragment feature includes methylated cfDNA fragments with fragment lengths of 61 bp, 62 bp, 63 bp, 64 bp, and 65 bp. For example, if the fragment feature is 74-75 bp, the fragment feature includes methylated cfDNA fragments with fragment lengths of 74 bp and 75 bp.

The frequency of the fragment feature refers to a ratio of a count of all methylated cfDNA fragments within the fragment feature to the total count of methylated cfDNA fragments. The fragment feature frequency profile includes a plurality of frequencies corresponding to all fragment features.

The distribution frequency of a terminal sequence refers to a ratio of a count of methylated cfDNA fragments having a same terminal sequence to the total count of methylated cfDNA fragments. The terminal sequence frequency profile includes a plurality of distribution frequencies corresponding to all terminal sequences.

In some embodiments, distribution frequencies of 4mer (e.g., CCGT, AGTT) to 6mer (e.g., CCGATC, TCGGAT) terminal sequences at 5′ end of the methylated cfDNA fragments are obtained.

In some embodiments, in the step S3, the candidate markers that exhibit significant differences between the cancer patients and the normal subjects are screened by a statistical method or a machine learning method. The machine learning method includes logistic regression, decision tree, random forest, support vector machine, naive Bayes, K-nearest neighbors, and neural network, or the like, or any combination thereof.

Merely by way of example, in the step S3, a non-parametric test, namely a Wilcoxon Rank-Sum Test, may be used to screen for significant features, from the candidate marker level profiles, that exhibit significant differences between the cancer patients and the normal subjects, such as specific genomic window features, fragment features, and terminal sequence features. Subsequently, a classification model, such as a logistic regression, is trained using 5-Fold Cross-Validation to evaluate a classification performance of each significant feature, with a performance metric being an Area Under the ROC Curve (AUC) value, and significant features having higher AUC values are selected as the candidate markers.

A screening process of the Wilcoxon rank-sum test may include the following operations. (a) Feature-by-feature testing: for each feature in the candidate marker level profile, such as a specific genomic window chr15: 31775701-31776000, a specific fragment feature 165-166 bp, or a specific terminal sequence ATGGGG, the Wilcoxon test is performed individually. (b) P-value calculation: for each feature, a p-value is calculated. A smaller p-value indicates that a difference between the features of the cancer patients and the normal subjects is more significant and is less likely to be caused by random factors. (c) Significance determination: a significance threshold (for example, α=0.05) is set. Features with a p-value smaller than the threshold are screened out as significant features, showing significant differences between the cancer patients and the normal subjects. Merely by way of example, an evaluation process of 5-fold cross-validation may include the following operations: (a) Data partitioning: a plurality of samples (for example, 181 cancer patients and 147 normal subjects) are randomly divided into five subsets (folds) of approximately equal size. (b) Iterative training and testing: five iterations are performed. In each iteration, 4-fold data are used as a training set of the classification model, and the remaining 1-fold data is used as a testing set of the classification model. (c) The AUC value calculation: on the testing set, a ROC curve is plotted based on prediction scores and true labels of the samples, and the AUC value is calculated. After five iterations, five AUC values (AUC₁, AUC₂, AUC₃, AUC₄, AUC₅) are obtained for each significant feature. (d) Result summarization: an average value of the five AUC values is calculated as a final evaluation metric of the discrimination capability of the feature. For example, an average AUC of feature chr15: 31775701-31776000 is 0.845. The above process is merely an exemplary description, and other statistical and/or machine learning methods may also be used to screen the candidate markers that exhibit significant differences between the cancer patients and the normal subjects.

In some embodiments, in the step S3, the machine learning method includes logistic regression, decision tree, random forest, support vector machine, naive Bayes, K-nearest neighbors, and neural network. That is, in the step S3, the candidate markers that exhibit significant differences between the cancer patients and the normal subjects are screened by any one or any combination of the logistic regression, the decision tree, the random forest, the support vector machine, the naive Bayes, the K-nearest neighbors, and the neural network.

In some embodiments, the present disclosure provides a system for screening markers for diagnosing cancer based on methylated cfDNA fragments, including: a methylation data input module, configured to receive the fragment sequence information and the methylation information of the cfDNA samples from the cancer patients and the cfDNA samples from the normal subjects; a genomic data storage module, configured to store whole human genome data; a data alignment module, connected to the methylation data input module and the genomic data storage module, respectively, and configured to align the fragment sequence information and the methylation information with the whole human genome data; an analysis module, connected to the data alignment module, and configured to perform the following analyses to obtain the candidate marker level profile: (a) dividing the whole human genome by length to obtain the plurality of different genomic windows, determining the count of methylated cfDNA fragments aligned to each of the plurality of different genomic windows, and obtaining the genomic window methylation level profile based on the methylation information and the count of methylated cfDNA fragments; (b) determining frequencies of fragment features with different step sizes based on counts of methylated cfDNA fragments of different lengths to obtain a fragment feature frequency profile; and (c) determining the distribution frequencies of 4mer to 6mer terminal sequences at 5′ end of the methylated cfDNA fragments, to obtain the terminal sequence frequency profile; the candidate marker level profile includes the genomic window methylation level profile, the fragment feature frequency profile, and the terminal sequence frequency profile; and a screening module, connected to the analysis module, and configured to screen the candidate markers that exhibit significant differences between the cancer patients and the normal subjects based on the candidate marker level profile.

In some embodiments, the methylation data input module is further configured to preprocess the fragment sequence information, the preprocessing includes:
(a) removing adapter sequences;
(b) filtering out low-quality sequences in which more than 40% of bases have a quality score lower than Q15.
(c) filtering out sequences containing more than 5 bases of N;
(d) filtering out sequences with a length shorter than 30 (overly short sequences); and
(e) trimming four bases at the fragment ends with an average quality score lower than Q20.

In some embodiments, the present disclosure provides a marker combination for diagnosing cancer, including markers obtained by the method or system for screening markers for diagnosing cancer based on methylated cfDNA fragments provided in the present disclosure.

In some embodiments, the present disclosure provides another marker combination for diagnosing cancer, including:
(a) at least one from a genomic window combination consisting of chr15: 31775701-31776000, chr7: 32467501-32467800, and chr8: 24771301-24771600;
(b) at least one from a fragment feature combination consisting of 165-166 bp, 163-165 bp, and 167-168 bp; and
(c) at least one from a terminal sequence combination consisting of ATGGGG, ATAGGC, and ATGAGG;
   the cancer is colorectal cancer;
   including:
(a) at least one from a genomic window combination consisting of chr7: 32467501-32467800, chr7: 32467801-32468100, and chr20: 21376801-21377100;
(b) at least one from a fragment feature combination consisting of 149-150 bp, 151-152 bp, and 153-154 bp; and
(c) at least one from a terminal sequence combination consisting of ATGAGC, ATGG, and ATAGCG;
   the cancer is liver cancer;
   including:
(a) at least one from a genomic window combination consisting of chr20: 43726801-43727100, chr6: 107955901-107956200, and chr19: 19650901-19651200;
(b) at least one from a fragment feature combination consisting of 279-280 bp, 277-279 bp, and 280-282 bp; and
(c) at least one from a terminal sequence combination consisting of CTAGGG, TTCAGC, and ATAGGC;
   the cancer is pancreatic cancer;
   including:
(a) at least one from a genomic window combination consisting of chr7: 32467501-32467800, chr6: 107955901-107956200, and chr2: 39187201-39187500;
(b) at least one from a fragment feature combination consisting of 163-164 bp, 165-166 bp, and 156-160 bp; and
(c) at least one from a terminal sequence combination consisting of AGGGAG, AGGGGA, and TGAAAC;
   the cancer is gastric cancer; or
   including:
(a) at least one from a genomic window combination consisting of chr3: 51740701-51741000, chr9: 140128201-140128500, and chr17: 7670401-7670700;
(b) at least one from a fragment feature combination consisting of 267-268 bp, 259-260 bp, and 268-270 bp; and
(c) at least one from a terminal sequence combination consisting of GGGAAC, GGGAGT, and GGGAAG;
   the cancer is lung cancer.

In some embodiments, a detection reagent and/or a device for the marker combination described above may be used to prepare a kit for diagnosing cancer.

In some embodiments, the present disclosure provides a computer device, including: a memory, configured to store a computer program; and a processor, configured to execute the computer program to implement the method for screening markers for diagnosing cancer based on methylated cfDNA fragments according to the present disclosure.

In some embodiments, the present disclosure provides a non-transitory computer-readable storage medium. The computer-readable storage medium stores a computer program, and the computer program is executed by a processor to implement the method for screening markers for diagnosing cancer based on methylated cfDNA fragments according to the present disclosure.

In some embodiments, the present disclosure provides a system for diagnosing cancer, including: a marker data input module, configured to input levels of a plurality of markers in the marker combination according to the present disclosure; and a cancer judgment module, connected to the marker data input module, and configured to judge whether a subject has cancer or is at risk of having cancer based on the levels of the plurality of markers in the marker combination. The levels of the plurality of markers are determined based on a cfDNA sample of the subject. The cfDNA sample of the subject may be processed in a manner similar to or as same as the manner in S1 and S2. The system for diagnosing cancer may judge whether the subject has cancer or is at risk of having cancer based on the levels of the plurality of markers in the marker combination by comparing with one or more preset thresholds or using a machine learning method similar to or as same as the machine learning method as described in S3. The one or more preset thresholds are related to at least one of the count of methylated cfDNA fragments aligned to the genomic window, the frequency of the fragment feature, or the distribution frequency of the terminal sequence.

In some embodiments, the marker combination includes at least one of the genomic window, the fragment feature, or the terminal sequence.

When the marker is the genomic window, the level of the marker refers to the methylation level, obtained based on the count of methylated cfDNA fragments aligned to the genomic window.

When the marker is the fragment feature, the level of the marker refers to the frequency of the fragment feature.

When the marker is a terminal sequence, the level of the marker refers to the distribution frequency of the terminal sequence.

In the present disclosure, the term “cancer” includes solid tumors and hematologic malignancies. The solid tumors may include a colorectal cancer, a pancreatic cancer, a prostate cancer, a squamous cell carcinoma, a basal cell carcinoma, an adenocarcinoma, a hidradenocarcinoma, a sebaceous carcinoma, a papillary carcinoma, a papillary adenocarcinoma, a cystadenocarcinoma, a medullary carcinoma, a bronchogenic carcinoma, a hepatocellular carcinoma, a cholangiocarcinoma, a choriocarcinoma, a renal carcinoma, a cervical carcinoma, a testicular carcinoma, a lung cancer, and a melanoma. The hematologic malignancies may include a leukemia, such as an acute lymphoblastic leukemia and an acute myeloid leukemia (including a myeloblast leukemia, a promyelocyte leukemia, a myelomonocytic leukemia, a monocytic leukemia, and an erythroleukemia), chronic leukemias (including a chronic myelogenous/granulocytic leukemia and a chronic lymphocytic leukemia), etc.

The experimental manners in the following embodiments, unless otherwise specified, are conventional manners. The instruments and equipment used in the following embodiments, unless otherwise specified, are standard laboratory instruments and equipment. The experimental materials used in the following embodiments, unless otherwise specified, are purchased from regular biochemical reagent suppliers.

### Embodiment 1: Screening Markers for Diagnosing Cancer Based on Methylated cfDNA Fragments

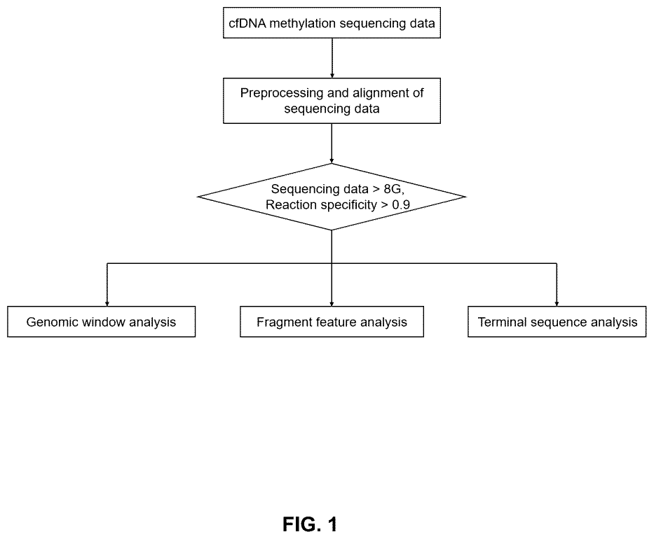

In conjunction with FIG. 1, this example provides a detailed description of a method for screening markers for diagnosing cancer based on methylated cfDNA fragments. FIG. 1 is a schematic flow diagram illustrating a method for screening markers for diagnosing cancer based on sequencing of methylated cfDNA fragments according to Embodiment 1 of the present disclosure.

### 1. Obtaining Methylation Sequencing Data

10 ng of human peripheral blood cfDNA, 10 μg of fully methylated positive control λDNA, and 10 μg of fully unmethylated negative control λDNA were mixed, followed by sequencing using methylated protein enrichment, immunoprecipitation, or enzymatic conversion manner. The sequencing platform was Illumina NovaSeq 6000.

After sequencing, raw sequencing data, namely the methylation sequencing data, were obtained.

### 2. Data Preprocessing

The raw sequencing data were preprocessed using fastp-0.20.0 software as follows:
(a) removing adapter sequences;
(b) filtering out low-quality sequences in which more than 40% of bases have a quality score lower than Q15;
(c) filtering out sequences containing more than 5 bases of N;
(d) filtering out sequences with a length shorter than 30 (overly short sequences), and
(e) trimming four bases at the fragment ends with an average quality score lower than Q20.

The data before and after preprocessing are shown in Table 1:

TABLE 1
Comparison of data before and after preprocessing

| | Before preprocessing | After preprocessing |
|---|---|---|
| Total count of reads | 137.91M | 135.20M |
| Total count of bases | 20.69G | 19.94G |
| Count of Q20 bases | 19.93G(96.34%) | 19.34G(96.98%) |
| Count of Q30 bases | 18.88G(91.24%) | 18.36G(92.08%) |
| GC content | 46.79% | 46.54% |

Preprocessing results are shown in Table 2:

TABLE 2
Preprocessing results

| | Count | Percentage |
|---|---|---|
| Reads after preprocessing | 135.20M | 98.03% |
| Low-quality reads removed | 2.48M | 1.80% |
| Reads containing excessive N removed | 147.61K | 0.11% |
| Reads with overly short sequences removed | 82.02K | 0.06% |

Next, the preprocessed sequence data were aligned to the human and λDNA reference genomes using the command-line software Bowtie2 (v2.3.4.2) to generate alignment files in SAM format. The SAM files were then sorted according to genomic coordinates using a samtools (v1.3.1) sort command to produce BAM files, followed by index construction using the samtools index command.

Subsequently, PCR-derived duplicates were removed using a Mark Duplicates command in Picard (v2.18.25).

Further, paired reads that were both aligned to the reference genomes with a MAPQ>20 were screened using a samtools view command.

Finally, a final alignment result was summarized, as shown in Table 3:

TABLE 3
Alignment Result

| | |
|---|---|
| Samples | 30602A101 |
| Count of reads aligned to the human reference genome | 135200508 |
| Count of duplicate reads aligned to the human reference | 46163345 |
| genome | |
| Total count of reads aligned to the human reference genome | 69495052 |
| after deduplication and screening | |
| Count of reads aligned to the positive control ADNA | 544928 |
| reference genome | |
| Count of reads aligned to the negative control ADNA | 53034 |
| reference genome | |

3. Calculating Effective Sequencing Data Yield and Reaction Specificity Rate of cfDNA in the Sample

The effective sequencing data yield of cfDNA in the sample was calculated based on the total count of reads aligned to the human reference genome after deduplication and screening (69,495,052×150/1024/1024/1000=9.941 G). The reaction specificity rate of cfDNA in the sample was determined based on the alignment result of the fully methylated positive control λDNA and the fully unmethylated negative control λDNA (544,928/(544,928+53,034)=0.911), as shown in Table 4:

TABLE 4
Effective sequencing data yield and reaction
specificity rate of cfDNA in the sample

| Sample | Effective sequencing data yield | Reaction specificity rate |
|---|---|---|
| 30602A101 | 9.941G | 0.911 |

### 4. Analysis of Insert Fragment Distribution

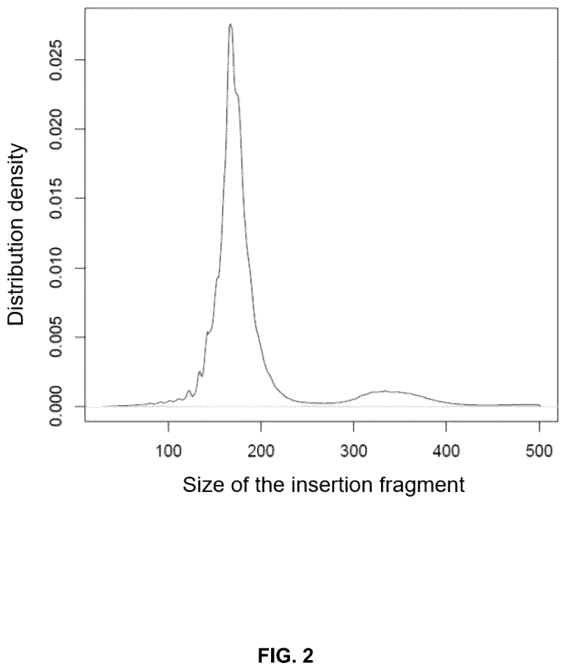

An insert fragment size analysis was performed based on the effective sequencing data yield of cfDNA in the sample. For all insert fragments aligned to the human reference genome after deduplication and screening, a length of each insert fragment was calculated, and distribution frequencies of insert fragments of different lengths were determined. Partial results are shown in Table 5, and the distribution frequencies of insert fragment sizes are illustrated in FIG. 2. FIG. 2 is a schematic diagram illustrating distribution frequencies of insert fragment size obtained through fragment size analysis according to Embodiment 1 of the present disclosure.

TABLE 5
Distribution of the insert fragments (partial)

| Size of | Frequency | Size of | Frequency | Size of | Frequency |
|---|---|---|---|---|---|
| the insert | of the | the insert | of the | the insert | of the |
| fragments | insert | fragments | insert | fragments | insert |
| (bp) | fragments | (bp) | fragments | (bp) | fragments |
| 60 | 2584 | 165 bp | 919548 | 220 | 36721 |
| 61 | 2686 | 166 bp | 960412 | 221 | 35178 |
| 62 | 2678 | 167 bp | 965259 | 222 | 32494 |
| 63 | 2754 | 168 bp | 948599 | 398 | 9268 |
| 64 | 2821 | 169 bp | 915977 | 399 | 8953 |
| 65 | 2988 | 170 bp | 872873 | 400 | 8404 |

Further, the insert fragment sizes of cfDNA in the sample were divided into fragment intervals with step lengths of 2 bp, 3 bp, 4 bp, 5 bp, . . . , and 10 bp, thereby partitioning cfDNA fragment lengths into different fragment intervals. When the step size is 2 bp, the divided fragment intervals are 61-62 bp, 63-64 bp, . . . , 399-400 bp. When the step size is 3 bp, the divided fragment intervals are 61-63 bp, 64-66 bp, . . . , and 397-399 bp. When the step size is 10 bp, the divided fragment intervals are 61-70 bp, 71-80 bp, . . . , 391-400 bp. The count of cfDNA fragments contained in each fragment interval was counted, representing the fragment features (i.e., features of insert fragment sizes). The proportion of cfDNA fragments in each fragment feature to the total cfDNA fragments was calculated to obtain the fragment feature frequency profile. Partial proportions of the fragment features are shown in Table 6:

TABLE 6
Distribution of the fragment features (partial)

| 2 | bp | Fragment interval | 61-62 bp | 63-64 bp | 65-66 bp |
|---|---|---|---|---|---|
| | | Proportion | 0.0001254076 | 0.0001289555 | 0.0001418076 |
| 3 | bp | Fragment interval | 61-63 bp | 64-66 bp | 67-69 bp |
| | | Proportion | 0.0001898491 | 0.0002063216 | 0.0002493671 |
| 4 | bp | Fragment interval | 61-64 bp | 65-68 bp | 69-72 bp |
| | | Proportion | 0.0002543631 | 0.0002982051 | 0.0003861424 |
| 5 | bp | Fragment interval | 61-65 bp | 66-70 bp | 71-75 bp |
| | | Proportion | 0.0003240903 | 0.0004207888 | 0.0005035492 |
| 6 | bp | Fragment interval | 61-66 bp | 67-72 bp | 73-78 bp |
| | | Proportion | 0.0003961707 | 0.0005425399 | 0.0006630963 |
| 7 | bp | Fragment interval | 61-67 bp | 68-74 bp | 75-81 bp |
| | | Proportion | 0.0004715456 | 0.0006678389 | 0.0009755654 |
| 8 | bp | Fragment interval | 61-68 bp | 69-76 bp | 77-84 bp |
| | | Proportion | 0.0005525682 | 0.0008070399 | 0.0011814525 |
| 9 | bp | Fragment interval | 61-69 bp | 70-78 bp | 79-87 bp |
| | | Proportion | 0.0006455378 | 0.0009562691 | 0.0013441131 |
| 10 | bp | Fragment interval | 61-70 bp | 71-80 bp | 81-90 bp |

| | | | |
|---|---|---|---|
| Proportion | 0.0007448791 | 0.0011871726 | 0.0015617301 |

### 5. Terminal Sequence Analysis

The terminal sequence analysis was performed based on the effective sequencing data yield of cfDNA in the sample. For all insert fragments aligned to the human reference genome after deduplication and screening, 4-6 base sequences at 5′ ends of the insert fragments were extracted, and distribution frequencies of 4mer and 6mer terminal sequences at 5′ ends of cfDNA were calculated to obtain the terminal sequence frequency profile. Partial results are shown in Table 7:

TABLE 7
Distribution frequencies of the terminal sequences (partial)

| 4mer terminal | | 6mer terminal | |
|---|---|---|---|
| sequence | Frequency | sequence | Frequency |
| AAAA | 0.007469 | AAAAAA | 0.001799 |
| AAAC | 0.002776 | AAAAAC | 0.000317 |
| AAAG | 0.00419 | AAAAAG | 0.00053 |
| AAAT | 0.004115 | AAAAAT | 0.000904 |
| AACA | 0.004115 | AAAACA | 0.000394 |
| AACA | 0.003117 | AAAACC | 0.000127 |

The methylation level analysis was performed based on the effective sequencing data yield of cfDNA in the sample. The methylation levels of any window in the whole human genome, including but not limited to 300 bp windows, were calculated to obtain the genomic window methylation level profile. In this example, methylation levels of 300 bp windows in CpG islands were calculated, as shown in Table 8:

TABLE 8
Methylation levels of 300 bp genomic windows in CpG islands

| Genomic window feature | FPKM value | Genomic window feature | FPKM value |
|---|---|---|---|
| chr1: 531001-531300 | 0.3503 | chr2: 3682801-3683100 | 2.4523 |
| chr1: 713101-713400 | 1.6349 | chr2: 3707101-3707400 | 1.9852 |
| chr1: 36787801-36788100 | 2.9194 | chr3: 71866501-71866800 | 0.8174 |
| chr1: 36807301-36807600 | 9.1087 | chr3: 72386401-72386700 | 1.7517 |
| chr2: 3452401-3452700 | 9.2255 | chr3: 73044901-73045200 | 2.8027 |
| chr2: 3504001-3504300 | 3.5033 | chr3: 75445501-75445800 | 7.0067 |
| chr2: 3520201-3520500 | 0.4671 | | |

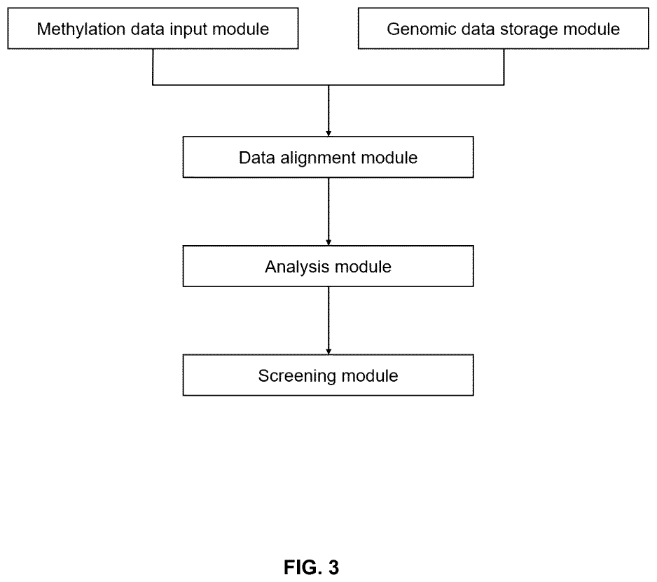

FIG. 3 is a schematic diagram illustrating a system for screening markers for diagnosing cancer based on sequencing of methylated cfDNA fragments according to Embodiment 1 of the present disclosure. Based on the above manners, this Embodiment also provides a system for screening markers for diagnosing cancer based on methylated cfDNA fragments, as shown in FIG. 3, including: a methylation data input module, configured to receive fragment sequence information and methylation information of cfDNA samples from cancer patients and cfDNA samples from normal subjects; a genomic data storage module, configured to store the whole human genome data; a data alignment module, connected to the methylation data input module and the genomic data storage module, respectively, and configured to align the cfDNA samples from the cancer patients and the cfDNA samples from the normal subjects with the whole human genome data; an analysis module, connected to the data alignment module, and configured to perform following analyses to obtain a candidate marker level profile: (a) obtaining counts of methylated cfDNA fragments in 300 bp genomic windows, to obtain a genomic window methylation level profile; (b) obtaining frequencies of fragment features with step sizes of 2-10 bp based on counts of methylated cfDNA fragments of different lengths, to obtain a fragment feature frequency profile; and (c) obtaining distribution frequencies of 4mer to 6mer terminal sequences at 5′ end of the methylated cfDNA fragments, to obtain a terminal sequence frequency profile; the candidate marker level profile includes the genomic window methylation level profile, the fragment feature frequency profile, and the terminal sequence frequency profile; and a screening module, connected to the analysis module, and configured to screen candidate markers that exhibit significant differences between the cancer patients and the normal subjects based on the candidate marker level profile.

### Embodiment 2: Application of Markers for Diagnosing Cancer Based on the Methylated cfDNA Fragments (Including Screening of Candidate Markers)

To verify the effectiveness of the manner in Embodiment 1 for identifying tumor diagnostic markers, the inventors collected blood samples from 181 colorectal cancer patients and 147 normal subjects. The cfDNA was extracted from peripheral blood, and the methylation sequencing data were obtained using the manner of Embodiment 1. The methylation sequencing data were then preprocessed to generate effective sequencing data, and the same manner was further applied to derive the genomic window methylation level profile, the fragment feature frequency profile, and the terminal sequence frequency profile of each sample.

Using the Wilcoxon rank-sum test combined with 5-fold cross-validation to calculate the AUC, colorectal cancer-specific enriched features were identified from the genomic window features, the fragment features, and the terminal sequence features, respectively. The results are shown in Table 9.

TABLE 9
Colorectal cancer-specific enriched features

| Genomic window feature | | Fragment feature | | Terminal sequence feature | |
|---|---|---|---|---|---|
| Feature name | AUC | Feature name | AUC | Feature name | AUC |
| chr15: 31775701-31776000 | 0.845 | 165-166 bp | 0.773 | ATGGGG | 0.778 |
| chr7: 32467501-32467800 | 0.843 | 163-165 bp | 0.763 | ATAGGC | 0.776 |
| chr8: 24771301-24771600 | 0.826 | 167-168 bp | 0.762 | ATGAGG | 0.771 |

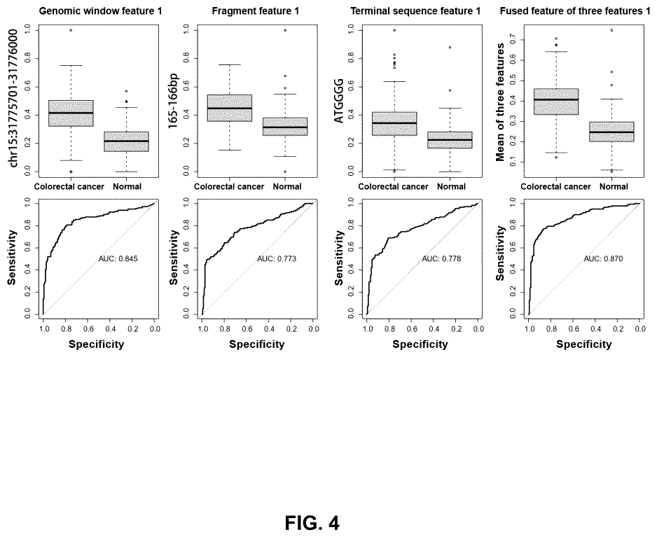

As shown in Table 9, the genomic window feature chr15: 31775701-31776000, the fragment feature 165-166 bp, and the terminal sequence feature ATGGGG achieved the highest AUC values for distinguishing colorectal cancer samples from normal subject samples (as shown in FIG. 4). FIG. 4 is a schematic diagram illustrating box plots showing distributions of a methylation level feature chr15: 31775701-31776000, a fragment feature 165-166 bp, and a terminal sequence feature ATGGGG highly enriched in the colorectum, in colorectal cancer samples and normal subject samples, and ROC curves illustrating an ability of the three features to distinguish the colorectal cancer samples from the normal subject samples. As shown in FIG. 4, the methylation level feature chr15: 31775701-31776000 distinguishes samples of the colorectal cancer and the normal subject with an AUC value of 0.845. The fragment feature 165-166 bp distinguishes samples of the colorectal cancer and the normal subject with an AUC value of 0.773. The terminal sequence feature ATGGGGG distinguishes samples of the colorectal cancer and the normal subject with an AUC value of 0.778. The fusion of the three features into a single feature distinguishes samples of the colorectal cancer and the normal subject with an AUC value of 0.87. Using mean of the min-max normalized values of the three features to fuse the three features into one feature, and using the fusion feature to differentiate the samples of the colorectal cancer and the normal subject boosted the AUC value to 0.87, which is a higher performance than using a feature of one dimension alone to differentiate the samples of the colorectal cancer and the normal subject (as shown in FIG. 4).

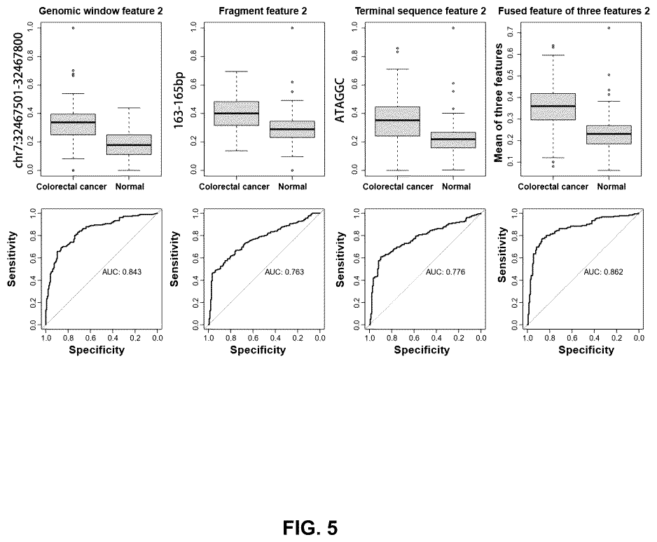

Similarly, the genomic window feature chr7: 32467501-32467800, a fragment feature 163-165 bp, and the terminal sequence feature ATAGGC differentiated the samples of the colorectal cancer and the normal subject with a second highest AUC (as shown in FIG. 5). FIG. 5 is a schematic diagram illustrating box plots showing distributions of a methylation level feature chr7: 32467501-32467800, the fragment feature 163-165 bp, and a terminal sequence feature ATAGGC highly enriched in the colorectum, in colorectal cancer samples and normal subject samples, and ROC curves illustrating an ability of the three features to distinguish the colorectal cancer samples from the normal subject samples. As shown in FIG. 5, the methylation level feature chr7: 32467501-32467800 distinguishes the samples of the colorectal cancer and the normal subject with an AUC value of 0.843. The fragment feature 163-165 bp distinguishes the samples of the colorectal cancer and the normal subject with an AUC value of 0.763. The terminal sequence feature ATAGGC distinguishes the samples of the colorectal cancer and the normal subject with an AUC value of 0.776. The fusion of the three features into a single feature distinguishes the samples of the colorectal cancer and the normal subject with an AUC value of 0.862. Using mean of the min-max normalized values of the three features to fuse the three features into one feature, and using the fusion feature to differentiate the samples of the colorectal cancer and the normal subject boosted the AUC value to 0.862, which is a higher performance than using a feature of one dimension alone to differentiate the samples of the colorectal cancer and the normal subject (as shown in FIG. 5).

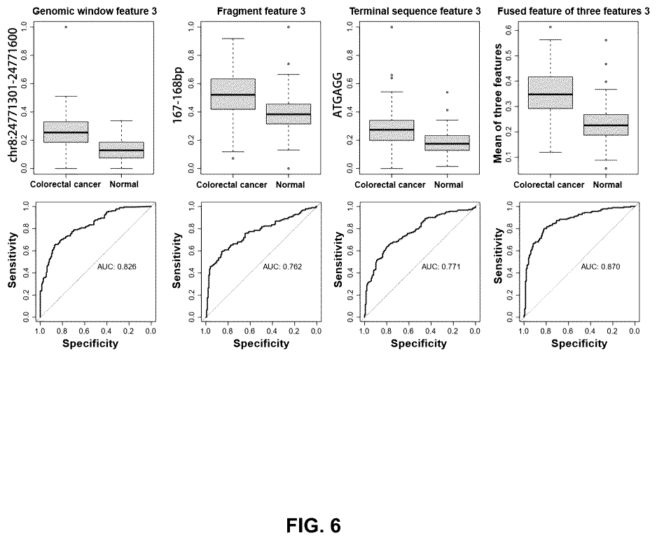

Similarly, the genomic window feature chr8: 24771301-24771600, a fragment feature 167-168 bp, and the terminal sequence feature ATGAGG differentiated the samples of the colorectal cancer and the normal subject with a third highest AUC (as shown in FIG. 6). FIG. 6 is a schematic diagram illustrating box plots showing distributions of a methylation level feature chr8: 24771301-24771600, a fragment feature 167-168 bp, and a terminal sequence feature ATGAGG highly enriched in the colorectum, in colorectal cancer samples and normal subject samples, and ROC curves illustrating an ability of the three features to distinguish the colorectal cancer samples from the normal subject samples. As shown in FIG. 6, the methylation level feature chr8: 24771301-24771600 distinguishes the samples of the colorectal cancer and the normal subject with an AUC value of 0.826. The fragment feature 167-168 bp distinguishes the samples of the colorectal cancer and the normal subject with an AUC value of 0.762. The terminal sequence feature ATGAGG distinguishes the samples of the colorectal cancer and the normal subject with an AUC value of 0.771. The fusion of the three features into a single feature distinguishes the samples of the colorectal cancer and the normal subject with an AUC value of 0.870. Using mean of the min-max normalized values of the three features to fuse the three features into one feature, and using the fusion feature to differentiate the samples of the colorectal cancer and the normal subject boosted the AUC value to 0.870, which is a higher performance than using a feature of one dimension alone to differentiate the samples of the colorectal cancer and the normal subject (as shown in FIG. 6).

Based on the above manners, this embodiment also provides a system for diagnosing cancer, including: a marker data input module, configured to input levels of a plurality of markers; and a cancer judgment module, connected to the marker data input module, and configured to judge whether a subject has cancer or is at risk of having cancer based on the levels of the plurality of markers in the marker combination.

### Embodiment 3: Performance of a Marker for Diagnosing Cancer Based on Methylated cfDNA Fragments in Other Cancers

To further validate the effectiveness of the method in Embodiment 1 for identifying diagnostic markers for cancers, blood samples were collected from 50 liver cancer patients, 123 pancreatic cancer patients, 75 gastric cancer patients, 57 lung cancer patients, and 142 healthy subjects. The cfDNA was extracted from peripheral blood, and the methylation sequencing data were obtained using the manner of Embodiment 1. The methylation sequencing data were then preprocessed to obtain the effective sequencing data, and the genomic window methylation level profiles, the fragment feature frequency profiles, and the terminal sequence frequency profiles of the samples were further obtained using the same method.

Using the same computational method in Embodiment 2, features specifically enriched in the liver cancer, the pancreatic cancer, the gastric cancer, and the lung cancer were identified from the genomic window features, the fragment features, and the terminal sequence features, respectively. The results are shown in Tables 10-13.

TABLE 10
Liver cancer-specific enriched features

| | | | | Terminal sequence | | Combination of the three |
|---|---|---|---|---|---|---|
| Genomic window feature | | Fragment feature | | feature | | features |
| Feature name | AUC | Feature name | AUC | Feature name | AUC | AUC |
| chr7: 32467501-32467800 | 0.941 | 149-150 bp | 0.957 | ATGAGC | 0.962 | 0.971 |
| chr7: 32467801-32468100 | 0.934 | 151-152 bp | 0.954 | ATGG | 0.958 | 0.969 |
| chr20: 21376801-21377100 | 0.915 | 153-154 bp | 0.949 | ATAGCG | 0.954 | 0.958 |

TABLE 11
Pancreatic cancer-specific enriched features

| | | | | Terminal sequence | | Combination of the three |
|---|---|---|---|---|---|---|
| Genomic window feature | | Fragment feature | | feature | | features |
| Feature name | AUC | Feature name | AUC | Feature name | AUC | AUC |
| chr20: 43726801-43727100 | 0.875 | 279-280 bp | 0.812 | CTAGGG | 0.865 | 0.901 |
| chr6: 107955901-107956200 | 0.856 | 277-279 bp | 0.809 | TTCAGC | 0.862 | 0.898 |
| chr19: 19650901-19651200 | 0.853 | 280-282 bp | 0.801 | ATAGGC | 0.862 | 0.895 |

TABLE 12
Gastric cancer-specific enriched features

| | | | | Terminal sequence | | Combination of the three |
|---|---|---|---|---|---|---|
| Genomic window feature | | Fragment feature | | feature | | features |
| Feature name | AUC | Feature name | AUC | Feature name | AUC | AUC |
| chr7: 32467501-32467800 | 0.910 | 163-164 bp | 0.797 | AGGGAG | 0.843 | 0.933 |
| chr6: 107955901-107956200 | 0.895 | 165-166 bp | 0.788 | AGGGGA | 0.836 | 0.914 |
| chr2: 39187201-39187500 | 0.893 | 156-160 bp | 0.788 | TGAAAC | 0.835 | 0.905 |

TABLE 13
Lung cancer-specific enriched features

| | | | | Terminal sequence | | Combination of the three |
|---|---|---|---|---|---|---|
| Genomic window feature | | Fragment feature | | feature | | features |
| Feature name | AUC | Feature name | AUC | Feature name | AUC | AUC |
| chr3: 51740701-51741000 | 0.956 | 267-268 bp | 0.978 | GGGAAC | 0.999 | 0.999 |
| chr9: 140128201-140128500 | 0.949 | 259-260 bp | 0.978 | GGGAGT | 0.999 | 0.999 |
| chr17: 7670401-7670700 | 0.948 | 268-270 bp | 0.977 | GGGAAG | 0.999 | 0.999 |

The beneficial effects of the embodiments of the present disclosure are as follows: (1) The method combines advanced methylated cfDNA sequencing technology and molecular biology manners, featuring high throughput, high sensitivity, and high accuracy. (2) In the method of the present disclosure, the methylated cfDNA fragments of the sample to be tested are first obtained by means of protein enrichment, immunoprecipitation, or enzymatic conversion. While capturing the methylated cfDNA fragments, the method effectively preserves the cfDNA fragment features, thereby enabling simultaneous detection of the cfDNA methylation levels, the insert fragment sizes, and the terminal sequences. This provides novel biomarkers and targets for disease diagnosis and therapy, offering broad application prospects.

All references cited in the present disclosure are incorporated herein by reference as if each reference is individually and specifically cited herein. Moreover, it should be understood that, after reading the foregoing description of the present disclosure, those skilled in the art may make various changes or modifications, and such equivalent forms likewise fall within the scope defined by the appended claims of the present specification.

What is claimed is:

1. A method for screening markers for diagnosing cancer based on methylated cfDNA fragments, comprising steps as follows:
S1, obtaining fragment sequence information and methylation information of cfDNA samples from cancer patients and cfDNA samples from normal subjects, wherein the methylation information includes methylation positions and methylation levels;
S2, performing the following analyses on the fragment sequence information and the methylation information obtained in the step S1 to obtain a candidate marker level profile:
(a) dividing a whole human genome by length to obtain a plurality of different genomic windows, determining a count of methylated cfDNA fragments aligned to each of the plurality of different genomic windows, and obtaining a genomic window methylation level profile based on the methylation information and the count of methylated cfDNA fragments;
(b) determining frequencies of fragment features with different step sizes based on counts of methylated cfDNA fragments of different lengths to obtain a fragment feature frequency profile; and
(c) determining distribution frequencies of 4mer to 6mer terminal sequences at 5′ end of the methylated cfDNA fragments to obtain a terminal sequence frequency profile;
wherein the candidate marker level profile includes the genomic window methylation level profile, the fragment feature frequency profile, and the terminal sequence frequency profile; and
S3, screening candidate markers that exhibit significant differences between the cancer patients and the normal subjects based on the candidate marker level profile obtained in the step S2, to determine markers for diagnosing cancer, including genomic windows, fragment features, and terminal sequences.

2. The method according to claim 1, wherein in the step S2, the genomic window methylation level profile is obtained based on steps as follows:
dividing the whole human genome by length to obtain the plurality of different genomic windows; for each genomic window among the plurality of different genomic windows, determining a count of methylated cfDNA fragments within the genomic window; and normalizing the count of methylated cfDNA fragments to obtain a methylation level of the genomic window, wherein a plurality of methylation levels corresponding to the plurality of different genomic windows constitute the genomic window methylation level profile.

3. The method according to claim 1, wherein in the step S3, the candidate markers that exhibit significant differences between the cancer patients and the normal subjects are screened by a statistical method or a machine learning method.

4. The method according to claim 3, wherein in the step S3, the machine learning method includes logistic regression, decision tree, random forest, support vector machine, naive Bayes, K-nearest neighbors, and neural network.

5. A system for screening markers for diagnosing cancer based on methylated cfDNA fragments, comprising:
a methylation data input module, configured to receive fragment sequence information and methylation information of cfDNA samples from cancer patients and cfDNA samples from normal subjects;
a genomic data storage module, configured to store whole human genome data;
a data alignment module, connected to the methylation data input module and the genomic data storage module, respectively, and configured to align the fragment sequence information and the methylation information with the whole human genome data;
an analysis module, connected to the data alignment module, and configured to perform the following analyses to obtain a candidate marker level profile:
(a) dividing a whole human genome by length to obtain a plurality of different genomic windows, determining a count of methylated cfDNA fragments aligned to each of the plurality of different genomic windows, and obtaining a genomic window methylation level profile based on the methylation information and the count of methylated cfDNA fragments;
(b) determining frequencies of fragment features with different step sizes based on counts of methylated cfDNA fragments of different lengths to obtain a fragment feature frequency profile; and
(c) determining distribution frequencies of 4mer to 6mer terminal sequences at 5′ end of the methylated cfDNA fragments to obtain a terminal sequence frequency profile;
wherein the candidate marker level profile includes the genomic window methylation level profile, the fragment feature frequency profile, and the terminal sequence frequency profile; and
a screening module, connected to the analysis module, and configured to screen candidate markers that exhibit significant differences between the cancer patients and the normal subjects based on the candidate marker level profile.

6. A marker combination for diagnosing cancer, comprising markers screened by the method according to claim 1.

7. A computer device, comprising:
a memory, configured to store a computer program; and
a processor, configured to execute the computer program to implement the method according to claim 1.

8. A non-transitory computer-readable storage medium, wherein:
the computer-readable storage medium stores a computer program, and the computer program is executed by a processor to implement the method according to claim 1.

9. A system for diagnosing cancer, comprising:
a marker data input module, configured to input levels of a plurality of markers in the marker combination according to claim 6; and
a cancer judgment module, connected to the marker data input module, and configured to judge whether a subject has cancer or is at risk of having cancer based on the levels of the plurality of markers in the marker combination.
